(19) United States
(12) Patent Application Publication
(10) Pub. No.: US 2006/0058615 A1
(43) Pub. Date: Mar. 16, 2006

(54) Method and system for facilitating surgery

(75) Inventors: Ajay Mahajan, Murphysboro, IL (US); Sumeer Lal, Greenwood, SC (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: SOUTHERN ILLINOIS UNIVERSITY, Carbondale, IL (US)

(21) Appl. No.: 11/198,561

(22) Filed: Jul. 15, 2005

Related U.S. Application Data
(63) Continuation of application No. 10/987,068, filed on Nov. 12, 2004.
(60) Provisional application No. 60/520,152, filed on Nov. 14, 2003.

(51) Int. Cl. A61B 5/05

(52) U.S. Cl. 600/407

(57) ABSTRACT

In a method for facilitating surgery, a signal or signals are transmitted from one or more transmitters coupled to a surgical probe, and the signal or signals are received at a plurality of receivers. An estimate of a position of a portion of the surgical probe is determined based on the signal or signals received by the plurality of receivers. An indication of the estimate of the position is displayed on a display unit, wherein the display unit displays a representation of an anatomy of a patient, and wherein the indication of the estimate of the position is integrated with the representation of the anatomy of the patient.

### CROSS-REFERENCES TO RELATED APPLICATIONS

This application is a continuation of U.S. patent application Ser. No. 10/987,068, filed Nov. 12, 2004, and entitled “METHOD AND APPARATUS FOR FACILITATING SURGERY, which claims the benefit of U.S. Provisional Patent Application No. 60/520,152, filed on Nov. 14, 2003, and entitled “METHOD AND APPARATUS FOR FACILITATING SURGERY.” Both of these applications are hereby incorporated by reference herein in their entireties for all purposes.

### BACKGROUND

Accurate image guided neurosurgery allows for minimal craniotomy (surgical removal of bone), smaller wounds, more accurate approaches to a target, etc. Targets may be tumors, blood clots, foreign objects (e.g., bullets), etc. There is a critical need for 3-dimensional (3D) position estimation systems that allow the surgeon to know with high accuracy the position of the probe-tip with respect to pre-operative magnetic resonance imaging (MR) or computer aided tomography (C) images on display in real time. Most systems that are currently being used in operation rooms for neurosurgery in most hospitals are stereoscopic camera based systems that track the surgeon's probe. These systems have several limitations including a resolution of about 2 mm (with up to 1 mm being the best possible under perfect working conditions), large space requirements, complex and time-consuming calibration schemes, and prone to occasional temporary or permanent failures. Temporary failures during surgery also often lead to abandoning the use of the system due to the recalibration complexities. Moreover, these systems typically cost about $500 K-$750 K and need a technician to oversee them during surgery.

During surgery, a position of a brain may shift, complicating efforts to accurately locate a probe-tip during neurosurgery. The position of the brain may shift during surgery because, for example, a release of pressure, repeated removal of tumor material from the brain, etc. Due to brain shifts, current position estimates from image guided systems often do not reflect the known position of tumors as seen in pre-operative MRI images. One approach that has been suggested is to use ultasonography registration on preoperative MR/CT images. These ultrasonic images are similar to the very popular “baby images” taken during pregnancy. The resolution of such images is usually not very good, with a typical resolution being around 5 mm.

In two systems known to the inventors, quick 3D ultrasonic scans can be superimposed in real-time with preoperative MRI images to help account for brain shifts during surgery. In one system, a camera is used to determine position and orientation of a surgical probe. In the other system, a camera is used to determine position and orientation of ultrasonography sensors with the help of four optical markers on the ultrasonography sensor probe.

### SUMMARY

In one embodiment, a method for facilitating surgery is provided. The method comprises transmitting a signal or signals from one or more transmitters coupled to a surgical probe, and receiving the signal or signals at a plurality of receivers. The method additionally comprises determining an estimate of a position of a portion of the surgical probe based on the signal or signals received by the plurality of receivers. The method also comprises displaying an indication of the estimate of the position on a display unit, wherein the display unit displays a representation of an anatomy of a patient, and wherein the indication of the estimate of the position is integrated with the representation of the anatomy of the patient.

In another embodiment, a system for facilitating surgery is provided The system comprises a surgical probe having one or more transmitters coupled thereto, the transmitter(s) configured to transmit a signal or signals. The system also comprises a plurality of receivers, and a position calculator operatively coupled to the plurality of receivers, wherein the position calculator is configured to determine an estimate of a position of a portion of the surgical probe based on the signal or signals received by the plurality of receivers. The system additionally comprises a display unit, and a display system operatively coupled to the position calculator and the display unit, wherein the display system is configured to cause the display unit to display a representation of an anatomy of the patient and an indication of the estimate of the position, wherein the indication of the estimate of the position is integrated with the representation of the anatomy.

In another aspect, another method for configuring a system for facilitating surgery is provided. The method comprises positioning a plurality of receivers in a desired configuration, and using a transmitter or transmitters and a position calculator coupled to the plurality of receivers to determine positions of the plurality of receivers. The method also comprises adjusting the position calculator using the determined positions of the plurality of receivers, and verifying an accuracy of the position calculator using a surgical probe having at least one transmitter coupled to the surgical probe.

### BRIEF DESCRIPTION OF THE DRAWINGS

FIG. 1 is a block diagram of an example system for determining a position of a transmitter in one dimension;

FIG. 2 is a block diagram of an example system for determining a position of a transmitter in two dimensions;

FIG. 3A is a block diagram of another example system for determining a position of a transmitter in two dimensions;

FIG. 3B is a plot showing signals generated by receivers of the system of FIG. 3A;

FIG. 4A is a block diagram of a system in which some receivers are away from a transmitter normal, and in which some receivers are positioned such that the receiver normal is at an angle to a line between the receiver and the transmitter,

FIG. 4B is a plot showing signals generated by receivers of the system of FIG. 4A;

FIG. 5A is a block diagram of a system in which receivers are at different distances from a transmitter;

FIG. 5B is a plot showing signals generated by receivers of the system of FIG. 5A;

FIG. 6 is a plot showing signals generated by two receivers;

FIG. 7 is a flow diagram of an example method for determining a position of a transmitter;

FIG. 8 is a plot showing envelopes of signals generated by two receivers;

FIG. 9 is a plot showing non-normalized envelopes and normalized envelopes of signals generated by two receivers;

FIG. 10 is a plot showing a normalized envelope of a signal generated by a receiver;

FIG. 11 is a plot showing corresponding peaks in signals generated by two receivers;

FIG. 12 is a block diagram of an example system for determining a position of a transmitter;

FIG. 13 is a plot of an example Optimum Cost Surface for a receiver configuration;

FIG. 14 is an example configuration of five receivers;

FIG. 15 is an example configuration of six receivers;

FIG. 16 is an example system that may be used to facilitate neurosurgery;

FIG. 17 is an example method for facilitating neurosurgery;

FIG. 18 is a block diagram of an example of the computing device depicted in FIG. 16; and

FIG. 19 is a flow diagram of an example method for determining a current position of a transmitter or probe.

### DETAILED DESCRIPTION

Determining Position

Embodiments of a method for determining a position of a transmitter will now be described. This method involves analyzing differences in times of arrival of a signal transmitted from a transmitter to a plurality of receivers. As will be described in more detail below, this method may be used to determine an estimate of a position of a portion of a surgical probe. It is to be understood that other techniques for determining a position of a transmitter, including known techniques, may be used as well to determine an estimate of a position of a portion of a surgical probe.

A system for determining a 1-dimensional (1D) position may comprise a single transmitter 20 at a position T moving in a straight line, and two receivers 22 and 24 at positions R1 and R2, respectively, as shown in FIG. 1. The difference in time of arrival (DTOA) between the two receivers may be used to estimate a 1D position of the transmitter with respect to the nearest receiver (i.e., a distance from the transmitter to the nearest receiver). The position T may change with time, and has coordinates (d, 0). ‘d’ may be the distance of the transmitter to the first receiver 22. ‘α’ may be the angle formed by the line that joins the two receivers and the horizontal axix x. ‘z’ may be the distance between the two receivers. ‘ΔT₁₂’ may be the DTOA between the transmitter and the two receivers. An equation for expressing the distance d for this 1D position system may be described as:

$$d = \frac{{z}^{2} - {c}^{2} Δ {T}_{12}^{2}}{2 ( c Δ {T}_{12} + z cos α )} \qquad ( 1 )$$

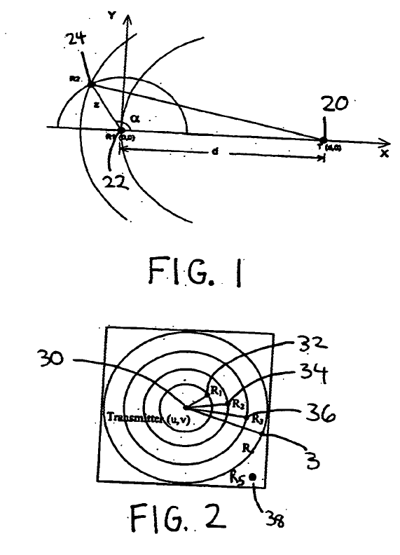

A system for determining 2-dimensional (2D) position may comprise a transmitter 30 and five receivers 32, 34, 36, 37, and 38 in a single plane, as shown in FIG. 2. The transmitter 30 may be located at a position (u, v). The receivers 32, 34, 36, 37, and 38 may be randomly located at known positions: R1 (x₁, y₁), R2 (x₂, y₂), R3 (x₃, y₃), R4 (X₄, y₄) and R5 (x₅, y₅), respectively. The time-of-flight between the transmitter 30 and any receiver may be unknown but the difference between times when the receivers sense the signals can be measured. In other words, if the transmitter 30 sends a signal at time T=0, the receivers 32, 34, 36, 37, and 38 will sense the signals at the unknown times T₁, T₂, T₃, T₄ and T₅, respectively. The DTOAs are then determined, which are: ΔT₁₂=T₂−T₁, ΔT₁₃=T₃−T₁, ΔT₁₄=T₄−T₁ and ΔT₁₅=T₅−T₁. A system of equations for determining an estimate of (u,v) may be:

$$[ \begin{matrix}2 ( {x}_{1} - {x}_{2} ) & 2 ( {y}_{1} - {y}_{2} ) & - 2 Δ {T}_{12} & - 2 Δ {T}_{12}^{2} \\ 2 ( {x}_{1} - {x}_{3} ) & 2 ( {y}_{1} - {y}_{3} ) & - 2 Δ {T}_{13} & - 2 Δ {T}_{13}^{2} \\ 2 ( {x}_{1} - {x}_{4} ) & 2 ( {y}_{1} - {y}_{4} ) & - 2 Δ {T}_{14} & - 2 Δ {T}_{14}^{2} \\ 2 ( {x}_{1} - {x}_{5} ) & 2 ( {y}_{1} - {y}_{5} ) & - 2 Δ {T}_{15} & - 2 Δ {T}_{15}^{2}\end{matrix} ] * [ \begin{matrix}u \\ v \\ cd \\ {c}^{2}\end{matrix} ] = [ \begin{matrix}{x}_{1}^{2} + {y}_{1}^{2} - {x}_{2}^{2} - {y}_{2}^{2} \\ {x}_{1}^{2} + {y}_{1}^{2} - {x}_{3}^{2} - {y}_{3}^{2} \\ {x}_{1}^{2} + {y}_{1}^{2} - {x}_{4}^{2} - {y}_{4}^{2} \\ {x}_{1}^{2} + {y}_{1}^{2} - {x}_{5}^{2} - {y}_{5}^{2}\end{matrix} ] \qquad ( 2 )$$

The above system of equations treats the speed of sound as a variable, and estimates it along with the position of the transmitter. The above analysis can be extended to a three-dimensional (3D) system having six receivers. In the 3D system, a system of equations for determining an estimate of a 3D position (u,v,w) may be described as:

$$[ \begin{matrix}2 ( {x}_{1} - {x}_{2} ) & 2 ( {y}_{1} - {y}_{2} ) & 2 ( {z}_{1} - 2 {z}_{2} ) & - 2 Δ {T}_{12} & - 2 Δ {T}_{12}^{2} \\ 2 ( {x}_{1} - {x}_{3} ) & 2 ( {y}_{1} - {y}_{3} ) & 2 ( {z}_{1} - 2 {z}_{3} ) & - 2 Δ {T}_{13} & - 2 Δ {T}_{13}^{2} \\ 2 ( {x}_{1} - {x}_{4} ) & 2 ( {y}_{1} - {y}_{4} ) & 2 ( {z}_{1} - 2 {z}_{4} ) & - 2 Δ {T}_{14} & - 2 Δ {T}_{14}^{2} \\ 2 ( {x}_{1} - {x}_{5} ) & 2 ( {y}_{1} - {y}_{5} ) & 2 ( {z}_{1} - 2 {z}_{5} ) & - 2 Δ {T}_{15} & - 2 Δ {T}_{15}^{2} \\ 2 ( {x}_{1} - {x}_{6} ) & 2 ( {y}_{1} - {y}_{6} ) & 2 ( {z}_{1} - 2 {z}_{6} ) & - 2 Δ {T}_{16} & - 2 Δ {T}_{16}^{2}\end{matrix} ] * [ \begin{matrix}u \\ v \\ w \\ cd \\ {c}^{2}\end{matrix} ] [ \begin{matrix}{x}_{1}^{2} + {y}_{1}^{2} + {z}_{1}^{2} - {x}_{2}^{2} - {y}_{2}^{2} - {z}_{2}^{2} \\ {x}_{1}^{2} + {y}_{1}^{2} + {z}_{1}^{2} - {x}_{3}^{2} - {y}_{3}^{2} - {z}_{3}^{2} \\ {x}_{1}^{2} + {y}_{1}^{2} + {z}_{1}^{2} - {x}_{4}^{2} - {y}_{4}^{2} - {z}_{4}^{2} \\ \begin{matrix}{x}_{1}^{2} + {y}_{1}^{2} + {z}_{1}^{2} - {x}_{5}^{2} - {y}_{5}^{2} - {z}_{5}^{2} \\ {x}_{1}^{2} + {y}_{1}^{2} + {z}_{1}^{2} - {x}_{6}^{2} - {y}_{6}^{2} - {z}_{6}^{2}\end{matrix}\end{matrix} ] \qquad ( 3 )$$

The above matrix equation can be written in the following vector form: where, A and B are known matrices and vector t is to be determined.

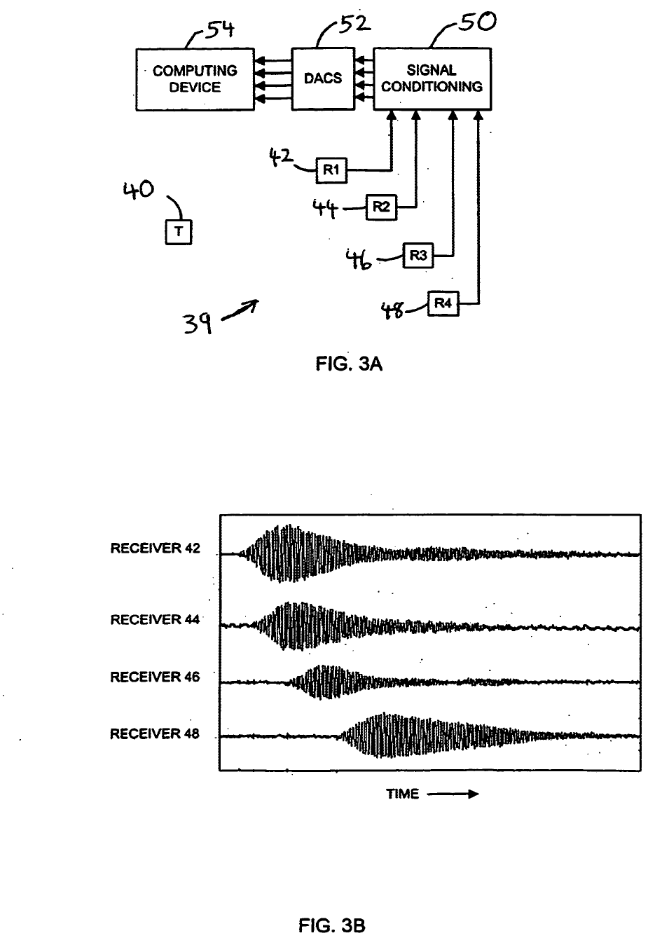

FIG. 3A is a block diagram illustrating one embodiment of a system 39 for determining a 2D position. The system comprises an ultrasonic transmitter 40, four receivers 42, 44, 46, and 48 at positions R1, R2, R3, and R4, respectively, a signal conditioning circuit 50, one or more digital-to-analog converters (DACs) 52, and a computing device 54. The computing device 54 may comprise, for example, one or more of a laptop, desktop, workstation, server, mainframe, a digital circuit, an analog circuit, an application specific integrated circuit (ASIC), a neural network, etc. In one example, the transmitter 40 emits signals at a frequency of 75 kHz and 10 cycles per burst. The received signals are conditioned, amplified, and may be sampled at rate of 150 MH. The conditioned, amplified, and sampled signals are illustrated in FIG. 3B. Other transmitter frequencies, different length bursts, and other sampling frequencies may also be used. The sampled signals may then be provided to the computing device 54. The DTOAs for the various receivers may then be calculated by the computing device 54. The computing device 54 may then determine a position of the transmitter based on the DTOAs. For example, a system of equations could be solved in which the speed of sound is considered a known value. Alternatively, a fifth receiver could be added, and equation (2) could be used to determine a position of the transmitter based on the DTOAS. The computing device 54 may thus act as a position calculator configured to determine an estimate of a position signals generated by the plurality of receivers. The computing device 54 may be so configured using software, hardware, and/or firmware.

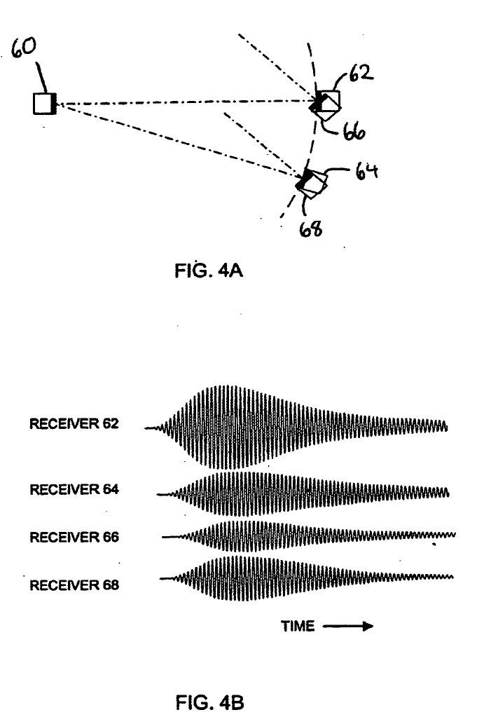

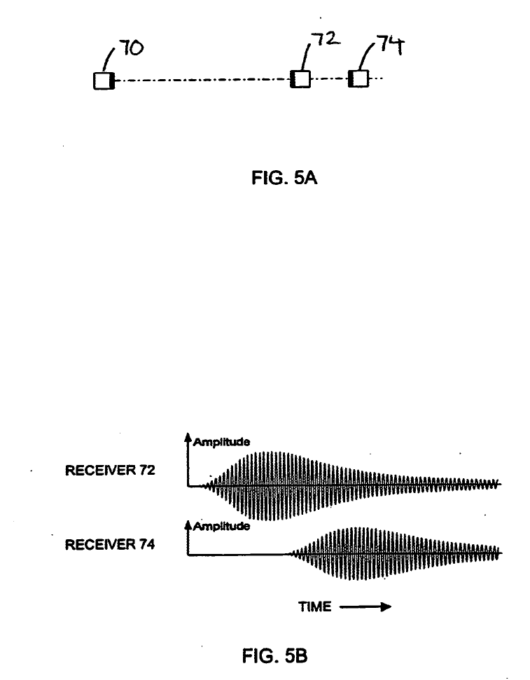

An overall signal amplitude produced by a transmitter-receiver pair may generally decrease as the receiver moves away from a transmitter normal, keeping the transmitter-receiver distance constant. For example, FIG. 4A illustrates a transmitter 60, a receiver 62 on the transmitter normal and a receiver 64 not on the transmitter normal, where the receivers 62 and 64 are at a substantially equal distance from the transmitter 60. As can be seen in FIG. 4B, the amplitude of the received signal generated by the receiver 64 is less than that generated by the receiver 62. Similarly, the overall signal amplitude decreases even further when a receiver normal makes an angle with a line joining the transmitter and receiver. In FIG. 4A, receiver normals of receivers 66 and 68 are at angles with respective lines between receivers 66 and 68 and the transmitter 60. As can be seen in FIG. 4B, the amplitudes of the received signal generated by the receivers 66 and 68 are less than that generated by the receivers 62 and 64. The overall signal amplitude produced by a transmitter-receiver pair also may generally decrease as the receiver moves away from the transmitter, as shown in FIGS. 5A and 5B. In FIG. 5A, a receiver 74 is at a greater distance from a transmitter 70 than a receiver 72. As can be seen in FIG. 5B, the amplitude of the received signal generated by the receiver 74 is less than that generated by the receiver 72. This is mainly due to the reason that the same energy from the transmitter is more spatially distributed as the receiver moves further away. Further, the sound energy may also be absorbed in a medium through which the sound energy travels.

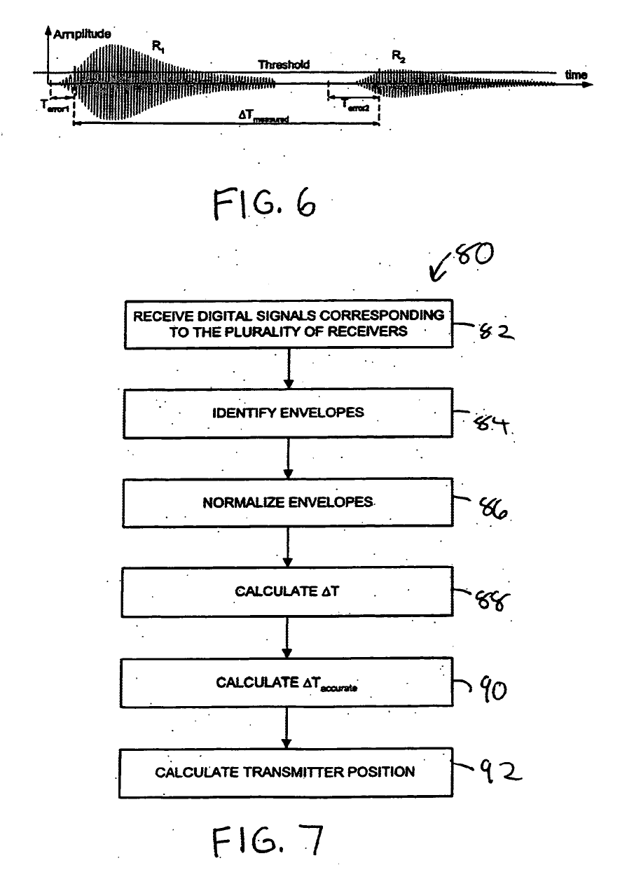

Any of a variety of techniques, including known techniques, may be used to determine the time of arrival of a wave at a receiver. For example, “thresholding” is one known method for signal detection, and may be used with a type of short duration signal. In this method, the received signal is compared with a threshold level, such that the arrival of the wave is acknowledged when the signal reaches this level. The threshold level is typically chosen in an attempt to eliminate or reduce false detections due to ground level noise, for example. The detection will occur at a time slightly after the signal was actually received. This leads to an error say Tₑᵣᵣₒᵣ. The error may not be able to be neglected if it is comparable to the time measurement. The Tₑᵣᵣₒᵣ, may be different for different receiver locations and misalignment, as shown in FIG. 6. So, even after taking the difference in time measurements, the errors may not cancel out. In other words, the term ΔTₑᵣᵣₒᵣ defined as follows may not be equal to zero:
ΔTₑᵣᵣₒᵣ=Tₑᵣᵣₒᵣ₂−Tₑᵣᵣₒᵣ₁ (5)
It will be understood that techniques other than thresholding, including known techniques, can be used to determine DTOAs.

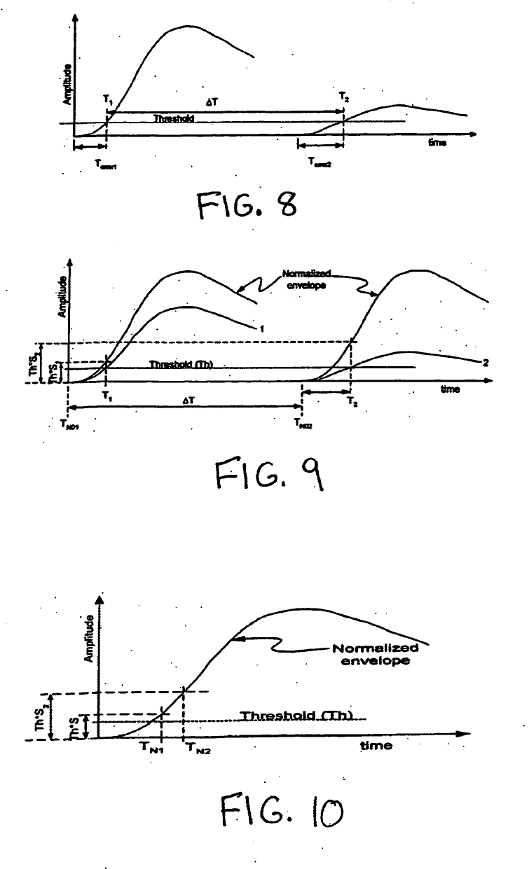

FIG. 7 is a flow diagram illustrating an example method 80 for determining a position of a transmitter based on DTOAs. The method 80 will be described with reference to FIG. 3A, and may be implemented, at least in part, by a computing device such as the computing device 54 of FIG. 3A. It will be understood, however, that the method 80 could be implemented by a system other than the system 39 of FIG. 3A.

At a block 82, digital signals corresponding to signals received by receivers (e.g., receivers 42, 44, 46, and 48) are received. At a block 84, envelopes in the signals received at the block 82 are identified. For example, a thresholding technique may be used to identify envelopes in signals received at receivers 42, 44, 46, and 48. At a block 86, the envelopes identified at the block 84 are normalized Δt a block 88, an estimate ΔT for each DTOAs is calculated. At a block 90, a more accurate estimate ΔT_{accurate} is calculated. Then, at a block 92, the transmitter position is calculated based on the ΔT_{accurate} values calculated at the block 90. Blocks 88, 90, and 92 will be described below in more detail.

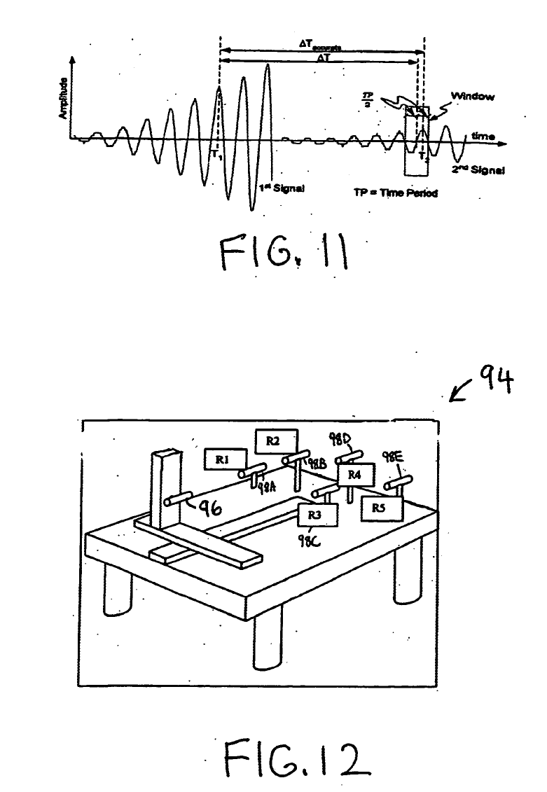

The calculation of ΔT will be described with reference to FIGS. 8-10. Experiments were conducted to capture signals from transmitter-receiver pair for different distances and misalignments. Envelopes of the signals were then determined. Referring to FIG. 8, times of arrivals T₁ and T₂ corresponding to two receivers were determined using a thresholding technique. It was observed that the errors in determining the beginnings of an envelope due to the thresholding technique may be different between the receivers. Thus, a first estimate ΔTₘ of the Difference in Time of Arrival (DTOA) determined by the difference between T₁ and T₂ may have an error ΔTₑᵣᵣₒᵣ defined by:
ΔTₑᵣᵣₒᵣ=Tₑᵣᵣₒᵣ₂−Tₑᵣᵣₒᵣ₁ (6)

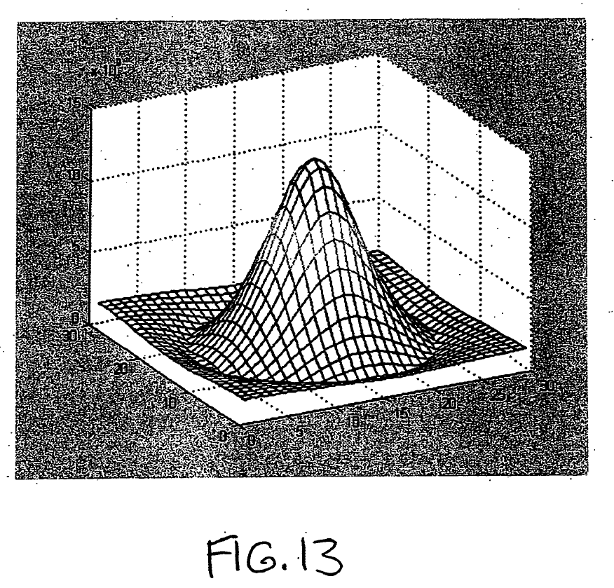

Determining an estimate of ΔTₑᵣᵣₒᵣ may help to generate a more accurate estimate of the DTOA. Let the envelope of the first and second signal cross the threshold (Th) at time T₁ and T₂ respectively. Let the signals 1 and 2 begin at T_{NO1} and T_{NO2}, respectively, and the scaling for normalization be S₁ and S₂ respectively, as shown in FIG. 9. A second estimate of DTOA, ΔT, of the two signals is:
ΔT=T_{N02}−T_{N01}=T₂−T₁+T_{N02}−T_{N01}−T₂+T₁
ΔT=ΔTₘ−(T₂−T_{N02})+(T₁−T_{N01}) (7)
where ΔTₘ=T₂−T₁ is the experimentally measured DTOA. Had normalization been done on both the signals, the threshold point of signal 1 and 2 would have had an amplitude of ‘Th*S₁’ and ‘Th*S₂’ respectively. The normalized signal, starting at t=0, is shown again in FIG. 10 for better understanding. It can be seen that:
T₂−T_{N02}=T_{N2}−0=T_{N2}; (8)
and
T₁−T_{N01}=T_{N1}−0=T_{N1} (9)
Thus, combining equation (7), (8) and (9), one gets:
ΔT=ΔTₘ−(T_{N2}−T_{N1})=ΔTₘ−ΔTₑᵣᵣₒᵣ (10)
where ΔTₑᵣᵣₒᵣ=T_{N2}−T_{N1} (11)

If the normalized signal can be approximated by a curve and, T_{N2} and T_{N1} can be determined from known ‘Th*S₁’ and ‘Th*S₂’, the problem of estimating ΔTₑᵣᵣₒᵣ may be solved. A 4^{th} or 5^{th} order polynomial, for example, can be used for curve fitting but in that case solving for time for a known amplitude will be a numerical analysis problem and hence, may be time consuming. Curve fitting with a sigmoid function is another possible technique that may be employed. The following function may be used for a maximum amplitude of 10V:

$$A = \frac{{K}_{1}}{1 + {ⅇ}^{- B ( t - C )}} - {K}_{2} \qquad ( 12 )$$

where A=Amplitude, t=time, K₁=10.6, B=29000, C=0.0001040, and K₂=0.3.

The equation may be valid from the initiation of the envelope till the peak. Rearranging equation (12), t can be determined from a known ‘A’:

$$t = C + \frac{1}{B} ln ( \frac{A + {K}_{2}}{{K}_{1} - {K}_{2} - A} ) \qquad ( 13 )$$

An interesting point to be noted is that with this approach it is not necessary to do the thresholding at a low value, as required by previous investigators, to minimize the error due to ΔTₑᵣᵣₒᵣ. Now multiple thresholding can be done to make multiple measurements at an instance. An average can be taken to get a more reliable measurement at each instant. It can be thought of as parallel measurement and then taking the average, instead of serial measurement and then taking the average.

Though the above mentioned procedure reduces the error (by as much as 60 μs in some situations), it was found experimentally that there still can be a residual error up to ±5.5 μs. A technique to further reduce the error is described below. After this reduction, the error may include errors that are hardware resolution dependent. For a pair of identical receivers, for each peak in the first receiver-signal there will be a corresponding peak in the second receiver-signal. The amplitude of the corresponding peak may be different based on the strengths of the ultrasonic waves absorbed by the two receivers. The time difference between the occurrences of these corresponding peaks may be used to determine the time taken by the wave front to sweep from the first receiver to the second and hence it may be a relatively accurate time measurement.

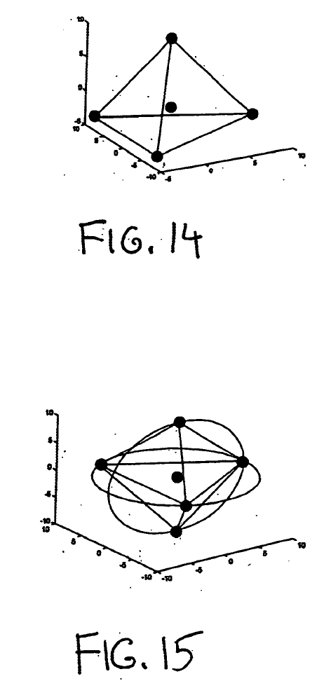

It has been experimentally verified that the corresponding peaks often exist, as shown in FIG. 11. Now, one example technique for identifying these peaks will be described. If one could estimate the ΔT within a range of half the time period on either side of the accurate ΔT, say ΔT_{accurate}, one could identify the corresponding peaks. In FIG. 11, the tail of the first signal is hidden for clarity. In one experiment, a 75 kHz ultrasonic wave was used, hence, the time period of the signal is 13.33 μs. Thus, even if one has an error of ±6.5 μs in ΔT from the previously described method, one can have a more accurate ΔT. Of course, this measurement can be still further improved. For example, a Kalman filter may be applied on the ΔT_{accurate}.

Two possible methods for finding the time difference between the corresponding peaks, among others, are windowing and phase difference. A peak can be selected in the first signal. Care should be taken to select this peak so that there exists a distinguishably amplified peak, with low noise in the second signal. Once a peak is selected, at T₁, in the first signal, a window can be created on the second signal for the time range (T₁+ΔT−0.5*TP) to (T₁+ΔT+0.5*TP), where ΔT is defined in equation (10) and TP is the time period of the signal, (13.33 μs for the example). There typically will be only one peak in this window, say at T₂. Thus,
ΔT_{accurate}=T₂−T₁ (14)

FIG. 12 illustrates another embodiment of a system 94 for determining a position of a transmitter. This system comprises a transmitter 96 and five receivers 98A-98E proximate to an area of interest.

The transmitter 96 may be located at an unknown position (u, v, w) which is of interest for some application, e.g., a portion of a surgical probe (e.g., a probe, scalpel, needle, etc.). The receivers 98A-98E may be located at known positions: R1 (x₁, y₁, zᵢ), R2 (x₂, y₂, z₂), R3 (x₃, y₃, z₃). R4 (x₄, y₄, z₅) and R5 (x₅, y₅, z₅). The transmitter 96 may transmit periodic signals which are received by the receivers. One receiver 98A will typically be the first to sense the signal and this receiver (R1) will be at a distance d from the transmitter 96. Another receiver 98B may be the second device to sense the signal and this device will be at a distance (d+cΔT₁₂), where c is the velocity of sound. The third, fourth, and fifth receivers 98C-9&E may be at distances (d+cΔT₁₃), (d+cΔT₁₄), and (d+cΔT₁₅) respectively from the transmitter 96. Since sound travels in spherical waves from the point source or transmitter 96, five concentric spheres can be drawn around the transmitter; one sphere of radius d through the point R₁; another sphere of radius d+cΔT₁₂ through the point R₂; a third sphere of radius d+cΔT₁₃ through the point R₃; a fourth sphere of radius d+cΔT₁₄ through the point R₄, and a fifth sphere of radius d+cΔT₁₄ through the point R₅. Writing equations for the spheres:

$$\begin{matrix}\begin{matrix}\begin{matrix}\begin{matrix}{( {x}_{1} - u )}^{2} + {( {y}_{1} - v )}^{2} + {( {z}_{1} - w )}^{2} = {d}^{2} \\ {( {x}_{2} - u )}^{2} + {( {y}_{2} - v )}^{2} + {( {z}_{2} - w )}^{2} = {( d + c Δ {T}_{12} )}^{2}\end{matrix} \\ {( {x}_{3} - u )}^{2} + {( {y}_{3} - v )}^{2} + {( {z}_{3} - w )}^{2} = {( d + c Δ {T}_{13} )}^{2}\end{matrix} \\ {( {x}_{4} - u )}^{2} + {( {y}_{4} - v )}^{2} + {( {z}_{4} - w )}^{2} = {( d + c Δ {T}_{14} )}^{2}\end{matrix} \\ {( {x}_{5} - u )}^{2} + {( {y}_{5} - v )}^{2} + {( {z}_{5} - w )}^{2} = {( d + c Δ {T}_{15} )}^{2}\end{matrix} \qquad ( 15 )$$

Multiplying out the equations and solving the first one for d² yields:
a^{a}=x₁²−2x₁u+u²+y₁²−2y₁v+v²+z₁²−2z₁w+w² (16)
Substituting d² into the remaining four equations gives four equations and four unknowns:

$$[ \begin{matrix}2 {x}_{1} - 2 {x}_{2} & 2 {y}_{1} - 2 {y}_{2} & 2 {z}_{1} - 2 {z}_{2} & - 2 c Δ {T}_{12} \\ 2 {x}_{1} - 2 {x}_{3} & 2 {y}_{1} - 2 {y}_{3} & 2 {z}_{1} - 2 {z}_{3} & - 2 c Δ {T}_{13} \\ 2 {x}_{1} - 2 {x}_{4} & 2 {y}_{1} - 2 {y}_{4} & 2 {z}_{1} - 2 {z}_{4} & - 2 c Δ {T}_{14} \\ 2 {x}_{1} - 2 {x}_{5} & 2 {y}_{1} - 2 {y}_{5} & 2 {z}_{1} - 2 {z}_{5} & - 2 c Δ {T}_{15}\end{matrix} ] * [ \begin{matrix}u \\ v \\ w \\ d\end{matrix} ] = [ \begin{matrix}{c}^{2} Δ {T}_{12}^{2} \\ {c}^{2} Δ {T}_{13}^{2} \\ {c}^{2} Δ {T}_{14}^{2} \\ {c}^{2} Δ {T}_{15}^{2}\end{matrix} \begin{matrix}+ {x}_{1}^{2} + {y}_{1}^{2} + {z}_{1}^{2} - {x}_{2}^{2} - {y}_{2}^{2} - {z}_{2}^{2} \\ + {x}_{1}^{2} + {y}_{1}^{2} + {z}_{1}^{2} - {x}_{3}^{2} - {y}_{3}^{2} - {z}_{3}^{2} \\ + {x}_{1}^{2} + {y}_{1}^{2} + {z}_{1}^{2} - {x}_{4}^{2} - {y}_{4}^{2} - {z}_{4}^{2} \\ + {x}_{1}^{2} + {y}_{1}^{2} + {z}_{1}^{2} - {x}_{5}^{2} - {y}_{5}^{2} - {z}_{5}^{2}\end{matrix} ] \qquad ( 17 )$$

which can be solved for the location of the transmitter (u, v, w) and the distance (d) from the first receiver (R₁) to the transmitter. The speed of sound may be expressed as an unknown, so that it can be estimated at every ranging operation. This may lead to a more accurate estimation of the coordinates of the transmitter since the local changes in the speed of sound do not lead to errors. The final formulation is given below:

$$[ \begin{matrix}2 ( {x}_{1} - {x}_{2} ) & 2 ( {y}_{1} - {y}_{2} ) & 2 ( {z}_{1} - {z}_{2} ) & - 2 Δ {T}_{12} & - 2 Δ {T}_{12}^{2} \\ 2 ( {x}_{1} - {x}_{3} ) & 2 ( {y}_{1} - {y}_{3} ) & 2 ( {z}_{1} - {z}_{3} ) & - 2 Δ {T}_{13} & - 2 Δ {T}_{13}^{2} \\ 2 ( {x}_{1} - {x}_{4} ) & 2 ( {y}_{1} - {y}_{4} ) & 2 ( {z}_{1} - {z}_{4} ) & - 2 Δ {T}_{14} & - 2 Δ {T}_{14}^{2} \\ 2 ( {x}_{1} - {x}_{5} ) & 2 ( {y}_{1} - {y}_{5} ) & 2 ( {z}_{1} - {z}_{5} ) & - 2 Δ {T}_{15} & - 2 Δ {T}_{15}^{2} \\ 2 ( {x}_{1} - {x}_{6} ) & 2 ( {y}_{1} - {y}_{6} ) & 2 ( {z}_{1} - {z}_{6} ) & - 2 Δ {T}_{16} & - 2 Δ {T}_{16}^{2}\end{matrix} ] * [ \begin{matrix}u \\ v \\ w \\ cd \\ {c}^{2}\end{matrix} ] = [ \begin{matrix}{x}_{1}^{2} + {y}_{1}^{2} + {z}_{1}^{2} - {x}_{2}^{2} - {y}_{2}^{2} - {z}_{2}^{2} \\ {x}_{1}^{2} + {y}_{1}^{2} + {z}_{1}^{2} - {x}_{3}^{2} - {y}_{3}^{2} - {z}_{3}^{2} \\ {x}_{1}^{2} + {y}_{1}^{2} + {z}_{1}^{2} - {x}_{4}^{2} - {y}_{4}^{2} - {z}_{4}^{2} \\ \begin{matrix}{x}_{1}^{2} + {y}_{1}^{2} + {z}_{1}^{2} - {x}_{5}^{2} - {y}_{5}^{2} - {z}_{5}^{2} \\ {x}_{1}^{2} + {y}_{1}^{2} + {z}_{1}^{2} - {x}_{6}^{2} - {y}_{6}^{2} - {z}_{6}^{2}\end{matrix}\end{matrix} ] \qquad ( 18 )$$

The above matrix equation can be written in the following vector form:
A*μ=B (19)
where, A and B are known matrices and vector t is to be determined. This formulation requires at least six receivers.
Singularity

For the 3D formulation with known speed of sound, the determinant of matrix A in equation (17) should not be zero for non-singularity. The following conditions should be satisfied to obtain a non-singular matrix. (1) All the five receivers should not lie on the same line, they should not even lie on a plane. This makes the first, second and the third column linearly dependent and thus the determinant becomes zero. (2) The projection of the receivers on xy, yz or zx plane should not lie on a line. This will produce linear dependency in two of the first three columns. (3) The first receiver and any two receivers should not lie on a line. When the transmitter is at the same position as the first receiver, two rows become linearly dependent. (4) All the five receivers should not lie on a sphere, the last column will be zero for the transmitter at the common center. This is the only singularity condition for the actual time of flights (TOFs) formulation and condition (1) is a special case of this when the radius is infinite.

Similar conditions exist for the other three formulations. There will be many other situations where the matrix A becomes singular for certain positions of the transmitter. An analytical solution for determining a set of receiver positions that will not result in a singularity for a set of possible transmitter positions has yet been developed. One possible technique for determining such a set of receiver positions is to conduct an exhaustive search for all or many of possible and/or likely transmitter positions for each receiver geometry using genetic algorithms (GAs). It is to be understood that other techniques may be used as well.

Choosing Locations by Using Genetic Algorithms

In one embodiment, a receiver geometry is selected such that the determinant of the matrix A is generally far away from zero for all potential transmitter positions in a given workspace. For the same relative receiver geometry, if the receiver distances are enlarged, the resolution and accuracy of the system will increase and hence the determinant of matrix A will also increase. The workspace may be taken as a square and a cube for the 2D and the 3D cases respectively so that they can be easily divided into smaller squares/cubes. The receivers may be placed within a fixed circle/sphere at the center of the square/cubic workspace. For a particular receiver geometry, the determinants may be calculated at the center of the small squares. In one embodiment, the minimum of the absolute value (MAV) of the determinants in the entire work space may be maximized. A configuration of the receiver can be selected as the one with the highest MAV.

A Genetic algorithm (GA) is a parallel, global search technique that simulates genetic reproduction and mutation in the natural selection process. A basic GA involves three types of operations: reproduction, crossover and mutation, which are repeatedly applied to a population of “chromosomes” or parameter strings.

Continuous parameter GA may be used to reduce the chromosome length and to avoid the Humming cliff diversion problem that is often encountered in binary parameter genetic algorithms. The chromosomes may be made up of x, y and z positions of all the receivers. The negative value of the MAV may be considered as the cost function (optimality criteria). Weighted random paring may be followed for selection, as cost weighting does not explore different regions. Offspring may be produced in two different ways for odd and even generations. Two-point random crossover may be used as it gives flexibility of two point center block crossover and one point crossover For even generations interior blending (simple crossover) may be done at the crossover point(s) and for the odd generations exterior blending (heuristic crossover) may be followed. This allows search inside and outside the range specified by the parameter value of parent crossover points. If the difference in cost of the best and worst eligible parent is less than, for example, 5% of cost of the best eligible parent, no offsprings are produced and evolution totally relies on mutation. This may be done, for example, because all of the almost same parents will not produce offspring any different than the parents. Thus, the chromosomes might be stuck at a point which may not even be a local minimum. In addition to all these techniques, a random search may also be done near the genes of the best chromosome in each generation. This may produce a better chromosome but the overall solution may get stuck in a local minimum and thus relies on the evolution of GA to move to better solution. The cost surface, as described earlier, may be the negative of the MAV. The negative sign is added since the GA has been developed to find the global minima Number of generations required to reach the global solution depends on the complexity of the problem. FIG. 13 shows an example Optimum Cost Surface for a receiver configuration.

It can be seen that the cost surface is a dome shape which reaches a minimum value before increasing again as the transmitter moves away from the origin. Knowledge of the cost surface in advance may help in the design of the positioning system.

Considering the problem of receivers in 3D with the transmitter also moving in a 3D space, one constraint that may be applied is that the speed of sound is known for a system. Such a system should include 5 receivers. One possible configuration for this formulation is a tetrahedron with an extra receiver added at the center of the tetrahedron (0,0,0). Such a configuration is illustrated in FIG. 14.

The last formulation to be considered is the same as the previous system with the known speed of sound constraint removed i.e., the sp of sound is unknown. One possible configuration for this formulation is three receivers on a sphere forming an equilateral triangle on a plane passing through the center of the sphere. Two more receivers are also on the sphere, the furthest point on the two sides of the plane. An additional receiver is added at the center of the tetrahedron (0,0,0), and the resulting final configuration is shown in FIG. 15. Hence, one configuration can be (0,0,10), (10,0,0), (−5,8.66,0), (−5,−8.66,0), (0,0,−10) and (0,0,0).

Having chosen a configuration, the next step is to install the receivers in this configuration. Generally, the receivers should be installed so that they are positioned an appropriate distance away from the surgical workspace so as to lessen the chance of a surgeon, a nurse, a technician, etc., accidentally moving the receivers during surgery. While installing the receivers, there will typically be some error in the positions of the receivers. Moreover, there will be situations when the receivers have been disturbed by accident or some other reason. An example procedure has been developed to use the same setup for installation and calibration, and is described below.

Installation and Calibration

Once a system is proposed, installation and calibration should be considered. Installation and calibration should be accurate because the subsequent accuracy of the system output depends on it, at least in part

As described previously, to estimate the location of the wave source, the receiver location should be known for the formation of the receiver location matrix A. Embodiments of the system may be used to find accurate locations of the receivers. In one embodiment, a method may be used to determine the location of the receivers by positioning a transmitter in various known locations. Installation and/or calibration may be performed by moving the transmitter to known coordinates of an inertial frame of reference, thereby defining the frame, and by measuring DTOAs among the receivers. This can be done, for example, by using a simple yet accurate 1D measuring device such as a ruler, linear slide, infrared or laser 1D system. Hence, one does not need an external expensive 3D position estimation system to measure the location of the receivers in 3D, but can use the system itself and measure the location with the accuracy of the system.

The second equation from equation (15) set may be subtracted from the first one:
2(x₂−x₁)u+2(y₂−y₁)v+2(z₂−z₁)w+(x₁²+y₁²+z₁²)−(x₂²+y₂²z₂²)+2cΔT₁₂d=−c²ΔT₁₂²
Let Δxᵢ=(xᵢ₋ₓ₁);
Δyᵢ=(yᵢ−y₁)
Δzᵢ=(zᵢ−z₁);
and rᵢ=(xᵢ²+yᵢ²+zᵢ²) for i=1, 2, 3, 4, 5 and 6.
Using these notations equation (20) can be rewritten as:
2Δx₂u+2y₂v+2z₂w+r₁²−r₂²+2cΔT₁₂d=−c₂ΔT₁₂² (21)
Five of these equations, for five values of (u,v,w) cannot be used to find Δx₂, Δy₂ and Δz₂, as d changes for every (u,v,w) hence, introducing a new unknown. Thus ‘d’ should be eliminated.

Subtracting the third equation from the first one in equation set (15) yields:
2Δx₃u+2y₃v+2z₃w+r₁²−r₃²+2cΔT₁₃=−c²ΔT₁₃² (22)
‘d’ can be eliminated by subtracting ΔT₁₂*Equation (22) from ΔT₁₃* Equation (21):
2ΔT₁₃uΔx₂−2ΔT₁₂uΔx₃+2ΔT₁₃vΔy₂−2ΔT₁₂vΔy₃+2ΔT₁₃wΔz₂−2ΔT₁₂wΔz₃+(r₁²−r₂²)ΔT₁₃−(r₁²−r₃²)ΔT₁₂=c²ΔT₁₃ΔT₁₂(ΔT₁₃−ΔT₁₂) (23)
This equation can be represented in matrix form for eight positions of the transmitter, (u,v,w):

$$[ \begin{matrix}Δ {T}_{131} {u}_{1} & Δ {T}_{121} {u}_{1} & Δ {T}_{131} {v}_{1} & Δ {T}_{121} {v}_{1} & Δ {T}_{131} {w}_{1} & Δ {T}_{121} {w}_{1} & Δ {T}_{131} & Δ {T}_{121} \\ Δ {T}_{132} {u}_{2} & Δ {T}_{122} {u}_{2} & Δ {T}_{132} {v}_{2} & Δ {T}_{122} {v}_{2} & Δ {T}_{132} {w}_{2} & Δ {T}_{122} {w}_{2} & Δ {T}_{132} & Δ {T}_{122} \\ Δ {T}_{133} {u}_{3} & Δ {T}_{123} {u}_{3} & Δ {T}_{133} {v}_{3} & Δ {T}_{123} {v}_{3} & Δ {T}_{133} {w}_{3} & Δ {T}_{123} {w}_{3} & Δ {T}_{133} & Δ {T}_{123} \\ Δ {T}_{134} {u}_{4} & Δ {T}_{124} {u}_{4} & Δ {T}_{134} {v}_{4} & Δ {T}_{124} {v}_{4} & Δ {T}_{134} {w}_{4} & Δ {T}_{124} {w}_{4} & Δ {T}_{134} & Δ {T}_{124} \\ Δ {T}_{135} {u}_{5} & Δ {T}_{125} {u}_{5} & Δ {T}_{135} {v}_{5} & Δ {T}_{125} {v}_{5} & Δ {T}_{135} {w}_{5} & Δ {T}_{125} {w}_{5} & Δ {T}_{135} & Δ {T}_{125} \\ Δ {T}_{136} {u}_{6} & Δ {T}_{126} {u}_{6} & Δ {T}_{136} {v}_{6} & Δ {T}_{126} {v}_{6} & Δ {T}_{136} {w}_{6} & Δ {T}_{126} {w}_{6} & Δ {T}_{136} & Δ {T}_{126} \\ Δ {T}_{137} {u}_{7} & Δ {T}_{127} {u}_{7} & Δ {T}_{137} {v}_{7} & Δ {T}_{127} {v}_{7} & Δ {T}_{137} {w}_{7} & Δ {T}_{127} {w}_{7} & Δ {T}_{137} & Δ {T}_{127} \\ Δ {T}_{138} {u}_{8} & Δ {T}_{128} {u}_{8} & Δ {T}_{138} {v}_{8} & Δ {T}_{128} {v}_{8} & Δ {T}_{138} {w}_{8} & Δ {T}_{128} {w}_{8} & Δ {T}_{138} & Δ {T}_{128}\end{matrix} ] \\ * \{ \begin{matrix}2 Δ {x}_{2} \\ - 2 Δ {x}_{3} \\ 2 Δ {y}_{2} \\ - 2 Δ {y}_{3} \\ 2 Δ {z}_{2} \\ - 2 Δ {z}_{3} \\ {r}_{1}^{2} - {r}_{2}^{2} \\ {r}_{3}^{2} + {r}_{1}^{2}\end{matrix} \} = {c}^{2} \{ \begin{matrix}Δ {T}_{131}^{2} Δ {T}_{121} - Δ {T}_{121}^{2} Δ {T}_{131} \\ Δ {T}_{132}^{2} Δ {T}_{122} - Δ {T}_{122}^{2} Δ {T}_{132} \\ Δ {T}_{133}^{2} Δ {T}_{123} - Δ {T}_{123}^{2} Δ {T}_{133} \\ Δ {T}_{134}^{2} Δ {T}_{124} - Δ {T}_{124}^{2} Δ {T}_{134} \\ Δ {T}_{135}^{2} Δ {T}_{125} - Δ {T}_{125}^{2} Δ {T}_{135} \\ Δ {T}_{136}^{2} Δ {T}_{126} - Δ {T}_{126}^{2} Δ {T}_{136} \\ Δ {T}_{137}^{2} Δ {T}_{127} - Δ {T}_{127}^{2} Δ {T}_{137} \\ Δ {T}_{138}^{2} Δ {T}_{128} - Δ {T}_{128}^{2} Δ {T}_{138}\end{matrix} \} \qquad ( 24 )$$

where (uₙ, vₙ, wₙ) is the coordinate of the transmitter in the n^{th} ranging operation and ΔTₗᵢₙ=DTOA between 1^{st} and i^{th} receiver in n^{th} ranging operation. The matrix equation (24) can be solved for (x₂−x₁), (x₃−x₁), (y₂−y₁), (y₃−y₁), (z₂−z₁), (z₃−z₁), (r₁²−r₂²) and (r₁²−r₃²) from eight ranging operations. Thus, the relative positions of second and third receiver have been found with respect to first receiver. The same procedure can be followed to find the relative position of any two, i^{th} and j^{th}, receivers with respect to the first receiver. The matrix equation for finding it follows directly from matrix equation (24) by substituting ‘i’ and ‘j’ for the second and third receivers respectively and the generalized matrix equation is:

$$[ \begin{matrix}Δ {T}_{1 j 1} {u}_{1} & Δ {T}_{1 i 1} {u}_{1} & Δ {T}_{1 j 1} {v}_{1} & Δ {T}_{1 i 1} {v}_{1} & Δ {T}_{1 j 1} {w}_{1} & Δ {T}_{1 i 1} {w}_{1} & Δ {T}_{1 j 1} & Δ {T}_{1 i 1} \\ Δ {T}_{1 j 2} {u}_{2} & Δ {T}_{1 i 2} {u}_{2} & Δ {T}_{1 j 2} {v}_{2} & Δ {T}_{1 i 2} {v}_{2} & Δ {T}_{1 j 2} {w}_{2} & Δ {T}_{1 i 2} {w}_{2} & Δ {T}_{1 j 2} & Δ {T}_{1 i 2} \\ Δ {T}_{1 j 3} {u}_{3} & Δ {T}_{1 i 3} {u}_{3} & Δ {T}_{1 j 3} {v}_{3} & Δ {T}_{1 i 3} {v}_{3} & Δ {T}_{1 j 3} {w}_{3} & Δ {T}_{1 i 3} {w}_{3} & Δ {T}_{1 j 3} & Δ {T}_{1 i 3} \\ Δ {T}_{1 j 4} {u}_{4} & Δ {T}_{1 i 4} {u}_{4} & Δ {T}_{1 j 4} {v}_{4} & Δ {T}_{1 i 4} {v}_{4} & Δ {T}_{1 j 4} {w}_{4} & Δ {T}_{1 i 4} {w}_{4} & Δ {T}_{1 j 4} & Δ {T}_{1 i 4} \\ Δ {T}_{1 j 5} {u}_{5} & Δ {T}_{1 i 5} {u}_{5} & Δ {T}_{1 j 5} {v}_{5} & Δ {T}_{1 i 5} {v}_{5} & Δ {T}_{1 j 5} {w}_{5} & Δ {T}_{1 i 5} {w}_{5} & Δ {T}_{1 j 5} & Δ {T}_{1 i 5} \\ Δ {T}_{1 j 6} {u}_{6} & Δ {T}_{1 i 6} {u}_{6} & Δ {T}_{1 j 6} {v}_{6} & Δ {T}_{1 i 6} {v}_{6} & Δ {T}_{1 j 6} {w}_{6} & Δ {T}_{1 i 6} {w}_{6} & Δ {T}_{1 j 6} & Δ {T}_{1 i 6} \\ Δ {T}_{1 j 7} {u}_{7} & Δ {T}_{1 i 7} {u}_{7} & Δ {T}_{1 j 7} {v}_{7} & Δ {T}_{1 i 7} {v}_{7} & Δ {T}_{1 j 7} {w}_{7} & Δ {T}_{1 i 7} {w}_{7} & Δ {T}_{1 j 7} & Δ {T}_{1 i 7} \\ Δ {T}_{1 j 8} {u}_{8} & Δ {T}_{1 i 8} {u}_{8} & Δ {T}_{1 j 8} {v}_{8} & Δ {T}_{1 i 8} {v}_{8} & Δ {T}_{1 j 8} {w}_{8} & Δ {T}_{1 i 8} {w}_{8} & Δ {T}_{1 j 8} & Δ {T}_{1 i 8}\end{matrix} ] * \{ \begin{matrix}2 Δ {x}_{i} \\ - 2 Δ {x}_{j} \\ 2 Δ {y}_{i} \\ - 2 Δ {y}_{j} \\ 2 Δ {z}_{i} \\ - 2 Δ {z}_{j} \\ {r}_{i}^{2} - {r}_{i}^{2} \\ {r}_{j}^{2} + {r}_{i}^{2}\end{matrix} \} = {c}^{2} \{ \begin{matrix}Δ {T}_{1 j 1}^{2} Δ {T}_{1 i 1} - Δ {T}_{1 i 1}^{2} Δ {T}_{1 j 1} \\ Δ {T}_{1 j 2}^{2} Δ {T}_{1 i 2} - Δ {T}_{1 i 2}^{2} Δ {T}_{1 j 2} \\ Δ {T}_{1 j 3}^{2} Δ {T}_{1 i 3} - Δ {T}_{1 i 3}^{2} Δ {T}_{1 j 3} \\ Δ {T}_{1 j 4}^{2} Δ {T}_{1 i 4} - Δ {T}_{1 i 4}^{2} Δ {T}_{1 j 4} \\ Δ {T}_{1 j 5}^{2} Δ {T}_{1 i 5} - Δ {T}_{1 i 5}^{2} Δ {T}_{1 j 5} \\ Δ {T}_{1 j 6}^{2} Δ {T}_{1 i 6} - Δ {T}_{1 i 6}^{2} Δ {T}_{1 j 6} \\ Δ {T}_{1 j 7}^{2} Δ {T}_{1 i 7} - Δ {T}_{1 i 7}^{2} Δ {T}_{1 j 7} \\ Δ {T}_{1 j 8}^{2} Δ {T}_{1 i 8} - Δ {T}_{1 i 8}^{2} Δ {T}_{1 j 8}\end{matrix} \} \qquad ( 25 )$$

Thus, the relative position of any number of receivers with respect to the first receiver can be found by just eight ranging operations. Now, to find absolute position of all the receivers, absolute position of the first receiver needs to be determined. It can be found as follows:

$$\begin{matrix}\begin{matrix}{r}_{1}^{2} - {r}_{2}^{2} = {x}_{1}^{2} + {y}_{1}^{2} + {z}_{1}^{2} - ( {x}_{2}^{2} + {y}_{2}^{2} + {z}_{2}^{2} ) \\ = ( {x}_{1}^{2} - {x}_{2}^{2} ) + ( {y}_{1}^{2} - {y}_{2}^{2} ) + ( {z}_{1}^{2} - {z}_{2}^{2} ) \\ = ( {x}_{1} - {x}_{2} ) ( {x}_{1} + {x}_{2} ) + ( {y}_{1} - {y}_{2} ) ( {y}_{1} + {y}_{2} ) + ( {z}_{1} - {z}_{2} ) ( {z}_{1} + {z}_{2} ) \\ = - Δ {x}_{2} ( {x}_{1} + Δ {x}_{2} + {x}_{1} ) - Δ {y}_{2} ( {y}_{1} + Δ {y}_{2} + {y}_{1} ) - Δ {z}_{2} ( {z}_{1} + Δ {z}_{2} + {z}_{1} )\end{matrix} & ( 26 ) \\ ∴ {r}_{1}^{2} - {r}_{2}^{2} = - \{ {( Δ {x}_{2} )}^{2} + {( Δ {y}_{2} )}^{2} + {( Δ {y}_{2} )}^{2} + 2 Δ {x}_{2} {x}_{1} + 2 Δ {y}_{2} {y}_{1} + 2 Δ {z}_{2} {z}_{1} \} \\ Similarly , & \\ ∴ {r}_{1}^{2} - {r}_{3}^{2} = - \{ {( Δ {x}_{3} )}^{2} + {( Δ {y}_{3} )}^{2} + {( Δ {y}_{3} )}^{2} + 2 Δ {x}_{3} {x}_{1} + 2 Δ {y}_{3} {y}_{1} + 2 Δ {z}_{3} {z}_{1} \} & ( 27 ) \\ ∴ {r}_{1}^{2} - {r}_{4}^{2} = - \{ {( Δ {x}_{4} )}^{2} + {( Δ {y}_{4} )}^{2} + {( Δ {y}_{4} )}^{2} + 2 Δ {x}_{4} {x}_{1} + 2 Δ {y}_{4} {y}_{1} + 2 Δ {z}_{4} {z}_{1} \} & ( 28 )\end{matrix}$$

Equation (26), (27) and (28) can be put into matrix form as:

$$[ \begin{matrix}2 Δ {x}_{2} & 2 Δ {y}_{2} & 2 Δ {z}_{2} \\ 2 Δ {x}_{3} & 2 Δ {y}_{3} & 2 Δ {z}_{3} \\ 2 Δ {x}_{4} & 2 Δ {y}_{4} & 2 Δ {z}_{4}\end{matrix} ] * \{ \begin{matrix}{x}_{1} \\ {y}_{1} \\ {z}_{1}\end{matrix} \} = \{ \begin{matrix}{r}_{2}^{2} - {r}_{1}^{2} - {( Δ {x}_{2} )}^{2} - {( Δ {y}_{2} )}^{2} - {( Δ {z}_{2} )}^{2} \\ {r}_{3}^{2} - {r}_{1}^{2} - {( Δ {x}_{3} )}^{2} - {( Δ {y}_{3} )}^{2} - {( Δ {z}_{3} )}^{2} \\ {r}_{4}^{2} - {r}_{1}^{2} - {( Δ {x}_{4} )}^{2} - {( Δ {y}_{4} )}^{2} - {( Δ {z}_{4} )}^{2}\end{matrix} \} \qquad ( 29 )$$

All the terms except x₁, y₁ and z₁ are known from the procedure described in the previous section. The above matrix equation can be solved for the absolute position of the first receiver. Hence, absolute position of all the other receivers can be determined. It is to be noted that four receivers are used to find the position of the first receiver using this procedure. Therefore it can be said that at least four receivers and eight ranging operations are required to absolutely define the coordinates of the receiver system using this procedure.

As discussed earlier, a minimum of eight-point measurements are required (with this procedure) to obtain the location of all the six or more receivers, but more points may be used for higher accuracy, in which case a least square estimation is done. This procedure gives the location of all the receivers simultaneously.

It is to be understood that the above-described calibration procedures are merely examples of procedures that may be used. Other procedures may be used as well.

Application to Surgery

Previous sections have described example techniques for 3D position estimation, conditions that help one to avoid singularities, example sensor placement configurations, and an example installation and calibration procedures. This section gives the application of an example system to real time image guided surgery. In particular, the example is in the context of neurosurgery. Similar systems can be used in other types of surgery as well, such as surgery on other organs and other parts of a body.

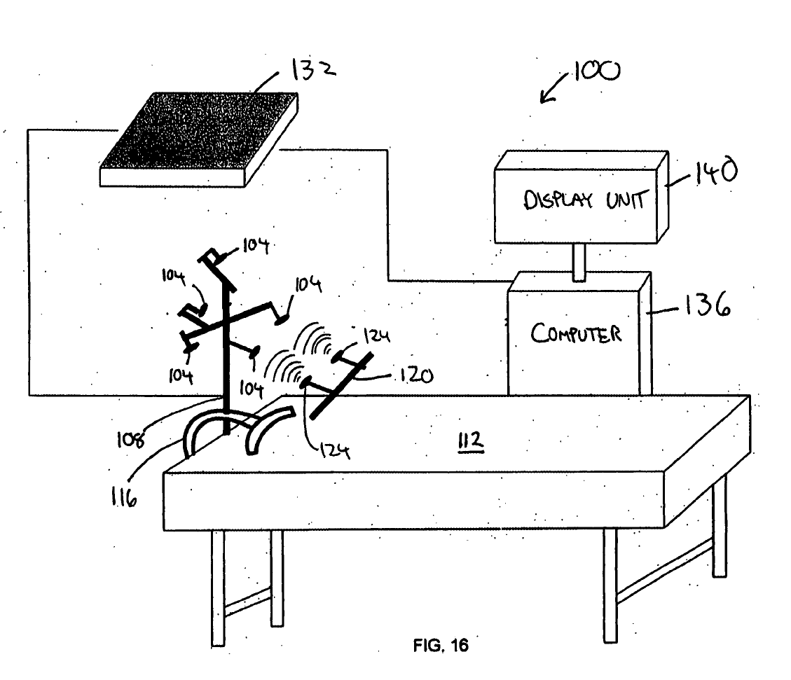

FIG. 16 is an illustration of an example system 100 that may be used to facilitate neurosurgery. The system 100 comprises a plurality of receivers 104 in a desired configuration. For example, the receivers 104 may be coupled to a stand 108. The system 100 may comprise 5, 6, 7, 8, or more receivers, for example. The system 100 also comprises an operating table 112 and a head constraint system 116. The stand 108 may be coupled to operating table 104 and/or the head constraint system 116. Similarly, the head constraint system 116 may be coupled to the operating table 104. The stand 108 may hold the receivers 104 in a desired configuration such as one of the configurations described above.

A surgical probe 120 (e.g., a surgical tool, a probe, a scalpel, a needle, etc.) may have coupled thereto two transmitters 124. By using two transmitters, a position of a portion of the probe (e.g., a tip or some other location on the probe) may be determined, using techniques such as those described above, without having to have a transmitter located at the portion of interest. For instance, a position of the tip of a probe may be determined without having to include a transmitter at the tip.

The receivers 104 may be operatively coupled to conditioning circuitry 132. The conditioning circuitry 132 may comprise, for example, filters, amplifiers, and one or more DACs. The conditioning circuitry 132 may be operatively coupled to a computing device 136. The computing device 136 may comprise, for example, one or more of a laptop, desktop, workstation, server, mainframe, a digital circuit, an analog circuit, an application specific integrated circuit (ASIC), a neural network, etc. The computing device 136 may be operatively coupled to a display unit 140 (e.g., a 2-dimensional or 3-dimensional display device).

In operation, the transmitters 124 may transmit signals, and the receivers 104 will receive the signals. The signal conditioning circuitry 132 may filter the received signals, amplify them, and convert the signals to digital values indicative of the received signals. Then, the digital values may be provided to the computing device 136. The computing device 136 may be configured (e.g., according to software, hardware, and/or firmware) to determine estimates of the two positions of the two transmitters 124 using techniques such as those described previously. Additionally, the computing device 136 may be configured to determine an estimate of a position of a portion of the surgical probe 120 based on the two determined positions. For instance, an estimate of a position of a tip of the probe may be determined based on the two position estimates. For example, the position of the tip may be estimated, at least in part, by determining a line that would pass through the two determined position estimates, and then estimating the tip position as a known distance from one of the transmitters along the determined line. The computing device 136 may thus act as a position calculator configured to determine an estimate of a position of the portion of the probe based on signals generated by the plurality of receivers. The computing device 136 may be so configured using software, hardware, and/or firmware.

Additionally, the computing device 136 may have a memory device (e.g., a hard disk drive) in which a representation of a patient's brain or spinal cord or a portion of the patient's-brain or spinal cord (e.g., a magnetic resonance imaging (MRI) scan, a computer aided tomography (C) scan, etc.) is stored. The computing device 136 may be configured to integrate the estimate of the position of the portion of the probe with the representation. Additionally, the computing device 136 may be configured to cause the display unit 140 to display the representation of the patient's brain or spinal cord as well as an indication of the estimated position of the surgical probe relative to the patient's brain or spinal cord. Such an integration and display can be accomplished using a variety of techniques, including known techniques.

Although computing device 136 is illustrated as a single device, the computing device 136 may comprise multiple devices. For example, one computing device may be used to determine the position of the portion of the surgical probe, and a separate computing device may be used to integrate the position of the portion of the probe with a representation of the patient's brain or spinal cord, and display the representation as well as an indication of the estimated position of the surgical probe relative to the patient's brain or spinal cord.

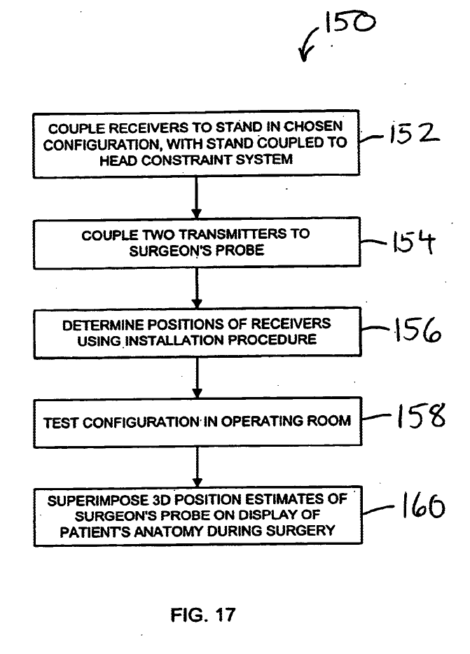

FIG. 17 is an example of a method 150 for facilitating neurosurgery. At a block 152, a plurality of receivers are coupled to a stand in a chosen configuration that is in turn coupled to a head constraint system. At a block 154, two transmitters are coupled to a surgeon's probe. For example, the transmitters may be externally coupled to the probe, embedded in the probe, etc. At a block 156, an installation procedure is used to determine the positions of the receivers in a lab setting, an operating room setting, etc. The positions determined at the block 156 can be used to adjust a position calculator that determines a position of a transmitter, probe, etc., based on DTOAs. At a block 158, the configuration may be tested in the operating room by positioning the surgeon's probe at known positions and/or employing known movements of the surgeon's probe, and comparing 3D position and/or movement estimates generated by the system with the known positions and/or movements. If an accuracy of the configuration is determined at the block 158 to be inadequate, the block 156 may be repeated. At a block 160, the 3D position estimates of the surgeon's probe generated by the system may be superimposed and/or integrated and/or mapped on a display of the patient's anatomy during surgery.

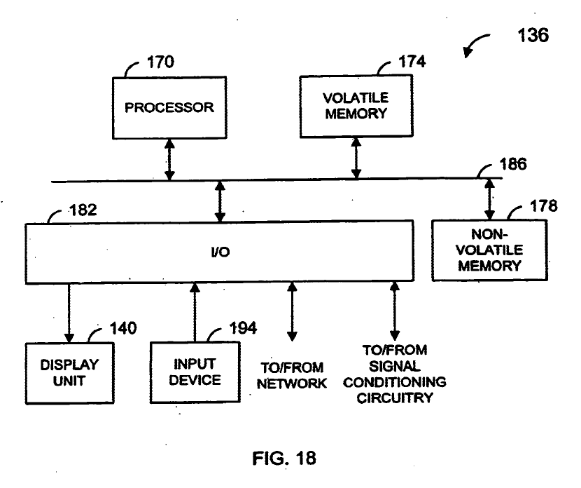

FIG. 18 is a block diagram of an example computing device 136 of FIG. 16. The computing device 54 of FIG. 3A may be the same or similar to the computing device 136. The computing device 136 may include at least one processor 170, a volatile memory 174, and a non-volatile memory 178. The volatile memory 174 may include, for example, a RAM, The non-volatile memory 178 may include, for example, one or more of a hard disk, a ROM, a CD-ROM, an EPROM, an EEPROM, a DVD, a FLASH memory, etc. The computer 136 may also include an 10 device 182. The processor 170, volatile memory 174, non-volatile memory 178, and I/O device 182 may be interconnected via one or more address/data buses 186. The computing device 136 may also include at least one user input device 194, The user input device 194 may include, for example, one or more of a keyboard, a keypad, a mouse, etc. In some embodiments, one or more of the volatile memory 174, non-volatile memory 178, and I/O device 182 may be coupled to the processor 170 via a bus separate from the address/data bus 186 (not shown), or coupled directly to the processor 170.

The display 140 and the user input device 194 are coupled to the I/O device 182. Additionally, the computing device 136 may be coupled to the signal conditioning circuitry 132 of FIG. 16. Further, the computing device 136 may be coupled to a network via the I/O device 182. Although the I/O device 182 is illustrated in FIG. 18 as one device, it may comprise several devices, Additionally, in some embodiments, one or more of the display 140, the user input device 194, and the signal conditioning circuitry 132 may be coupled directly to the address/data bus 186 or the processor 170. In one embodiment, the signal conditioning circuitry 132 may be enclosed, at least partially, within a housing of the computing device 132. For example, at least a portion of the signal conditioning circuitry 132 may be included on one or more expansion cards in the computing device 136. Alternatively, all of the signal conditioning circuitry 132 may be located externally to a housing of the computing device 132 and may be electrically and/or communicatively coupled to the computing device 132.

A representation of an anatomy of a patient may be stored, for example, in the non-volatile memory 178. The representation of an anatomy may be transferred to the non-volatile memory 178 via the network, via a communication link (not shown) to another computing system, via a CD-ROM, etc. Software instructions for implementing one or more of position detecting, installation/calibration, testing, superimposing/integrating of the position with the representation of the anatomy may be stored, in whole or in part, in the non-volatile memory 178, and executed, in whole or in part, by the processor 50.

Determining Positions Over Time

The position(s) of the transmitter(s) and/or surgical probe may be generated periodically at a rate that provides a surgeon with adequate position information. The rate will typically depend on the context in which a system is being used. A typical range of example rates at which the position(s) may be generated is 10-100 Hz. In some implementations, a rate less than 10 Hz may be adequate. In other implementations, a rate greater than 100 Hz may be required. Further, positions need not be determined at a constant rate. For example, the rate at which positions are determined may vary over time.

In one embodiment, each position of a transmitter and/or surgical probe may be generated based on a current set of measurements, and previous estimates and/or measurements may be ignored. In another embodiment, each position of the transmitter(s) and/or surgical probe may be generated based on a current set of measurements and based on previous estimates and/or measurements.

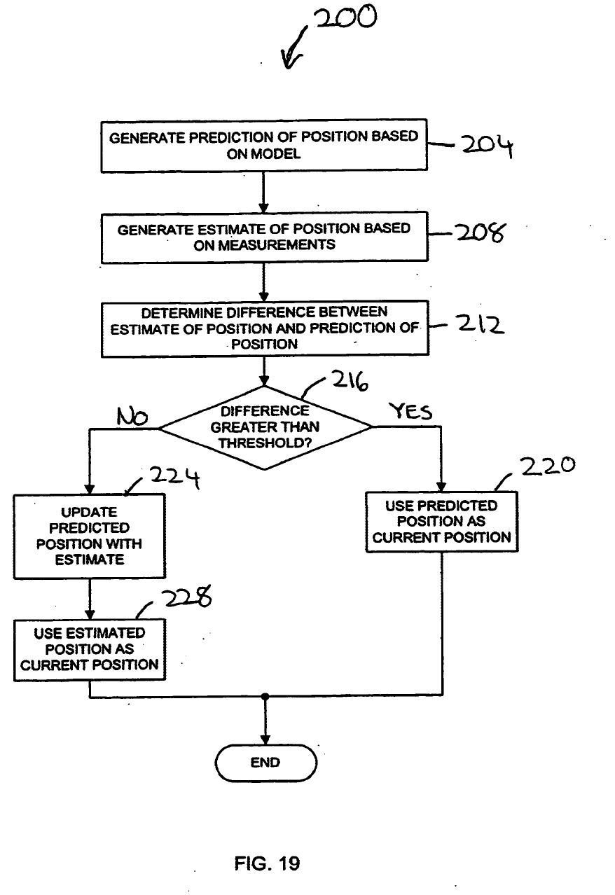

FIG. 19 is a block diagram of an example method 200 for generating a position of a transmitter and/or surgical probe based on previous position information. At a block 204, a prediction of the current position may be generated based on a model that models movement of the transmitter and/or surgical probe. Such a model will be described in more detail below. At a block 208, an estimate of the current position may be generated based on measured DTOAs. At a block 212, a difference between the prediction generated at the block 204 and the estimate generated at the block 208 is determined. Then, at a block 216, it is determined if the difference is greater than a threshold.

If the difference determined at the block 212 is greater than the threshold, the flow may proceed to a block 220. At the block 220, the prediction generated at the block 204 may be used as the current position, and the estimate generated at the block 208 may be ignored. Referring back to the block 216, if the difference determined at the block 212 is less than the threshold, the flow may proceed to a block 224. At the block 224, the measured estimate generated at the block 208 may be used to update the prediction generated at the block 204. At a block 228, the measured estimate generated at the block 208 may be used as the current position. Alternatively, the updated prediction generated at the block 224 may be used as the current position.

In one embodiment, the movement of the transmitter or surgical probe may be modeled using a state model. On example model of the movement of the transmitter or probe may be expressed as:
Xₖ₊₁=ΦₖXₖ+Wₖ (30)
where Xₖ is the state of the model at a time k, Xₖ₊₁ is a prediction of the model at a time k+1, and Φₖ is a matrix that may be determined based on experimental measurements using a variety of techniques, including known techniques. Wₖ is a vector of process noise values, where:
E{WₖW^{T}ᵢ}=Qₖ i=k Process Noise (White noise)
0i*k (31)
The state variable X may be a vector comprising one or more of a position, a velocity, an acceleration, etc., of the transmitter or probe. Referring to FIG. 19, equation 30 may be used to generate the prediction of a position at the block 204.

The measured DTOAs can also be modeled as being based on the state variable Xₖ:
Zₖ=HₖXₖ+Vₖ (32)
where Zₖ is the measured DTOAs at k and Hₖ is a matrix that may be determined based on experimental measurements using a variety of techniques, including known techniques. Additionally, Vₖ is noise where:

$$\begin{matrix}E \{ {V}_{k} {V}^{T} i \} = {R}_{k} & i = k \text{Measurement Noise} ( \text{White Noise} ) \\ 0 & i \neq k\end{matrix} \qquad ( 33 )$$

In view of equation (32), the state variable Xₖ can be iteratively adjusted according to the equation:
\hat{X}ₖ^{new}=\hat{X}ₖ^{old}+Kₖ(Zₖ−Hₖ\hat{X}ₖ^{old}) (34)
where \hat{X}ₖ^{new} is a new adjusted estimate of Xₖ, \hat{X}ₖ^{old} is the previous adjusted estimate of Xₖ, and Kₖ is a Kalman gain matrix that may be determined based on Experimental measurements using a variety of techniques, including known techniques. Initially, \hat{X}ₖ^{old} may be assumed to be a vector of zero values or some other values. Then, equation (34) can be applied repeatedly until it is determined that a convergence has occurred. For example, the iterative process may be stopped after the value Kₖ(Zₖ−Hₖ\hat{X}ₖ^{old}) falls below a threshold for some number of iterations (e.g., 1, 2, 3, 4, 5, 6, etc., iterations). After it is determined that the iterative process should be stopped, the value Xₖ can be set to \hat{X}ₖ^{new}. Referring again to FIG. 19, an iterative application of equation (34) can be used to update the predicted position at the block 224.

The above described technique is only one example technique that can be used to determine a position of a transmitter or probe based on a current set of measurements and previous sets of measurements. Other techniques may be used as well including, for example, using other types of models, other types of filters, neural networks, fuzzy logic, etc.

In some of the above-described examples, the transmitter or transmitters are described as transmitting ultrasound signals at particular frequencies and in bursts of particular lengths. It is to be understood that other types of ultrasound signals may be employed as well including, for example, signals having different frequencies, burst lengths, amplitudes, etc. Moreover, signals other than ultrasound signals may be employed including, for example, radio frequency signals, infrared signals, etc. Additionally, although particular sampling frequencies of DACs are described above, other sampling frequencies may be utilized.

Further, although particular numbers of transmitters and receivers are described above, some systems may utilize different numbers of transmitters and receivers. For example, different embodiments may utilized only one transmitter or more than two transmitters. Similarly, different embodiments may utilize more receivers than described above.

Software programs may be used to implementing some of the above-described methods, either in whole or in part. Such programs are for execution by a processor and may be embodied in software stored on a tangible medium such as CAROM, a floppy disk, a hard drive, a digital versatile disk (DVD), or a memory associated with the processor, but persons of ordinary skill in the art will readily appreciate that the entire program or parts thereof could alternatively be executed by a device other than a processor, and/or embodied in firmware and/or dedicated hardware in a well known manner.

While the invention is susceptible to various modifications and alternative constructions, certain illustrative embodiments thereof have been shown in the drawings and are described in detail herein. It should be understood, however, that there is no intention to limit the disclosure to the specific forms disclosed, but on the contrary, the intention is to cover all modifications, alternative constructions and equivalents falling within the spirit and scope of the disclosure as defined by the appended claims.

What is claimed is:

1. A method for facilitating surgery, the method comprising the acts of:
transmitting a first signal from a first transmitter coupled to a surgical probe;
receiving the first signal at a plurality of receivers;
determining an estimate of a position of a portion of the surgical probe based on the first signal received by the plurality of receivers;
displaying an indication of the estimate of the position on a display unit, wherein the display unit displays a representation of an anatomy of a patient, and wherein the indication of the estimate of the position is integrated with the representation of the anatomy of the patient.

2. A method as defined in claim 1, further comprising:
transmitting a second signal from a second transmitter coupled to the surgical probe, the second transmitter spaced apart from the first transmitter,
receiving the second signal at the plurality of receivers;
wherein determining the estimate of the position is further based on the second signal received by the plurality of receivers.

3. A method as defined in claim 1, wherein the plurality of receivers comprises a first receiver and a second receiver, wherein determining the estimate of the position comprises:
determining a first time difference between a time at which the first signal is received by the first receiver and a time at which the first signal is received by the second receiver; and
determining the estimate of the position based on the first time difference.

4. A method as defined in claim 3, wherein the plurality of receivers comprises a third receiver and a fourth receiver, wherein determining the estimate of the position comprises:
determining a second time difference between a time at which the first signal is received by the first receiver and a time at which the first signal is received by the third receiver;
determining a third time difference between a time at which the first signal is received by the first receiver and a time at which the first signal is received by the fourth receiver,
determining a fourth time difference between a time at which the first signal is received by the second receiver and a time at which the first signal is received by the third receiver;
determining a fifth time difference between a time at which the first signal is received by the second receiver and a time at which the first signal is received by the fourth receiver,
determining a sixth time difference between a time at which the first signal is received by the third receiver and a time at which the first signal is received by the fourth receiver, and
determining the estimate of the position further based on the second time difference, the third time difference, the fourth time difference, the fifth time difference, and the sixth time difference.

5. A method as defined in claim 4, wherein the plurality of receivers comprises a fifth receiver, wherein determining the estimate of the position comprises:
determining a seventh time difference between a time at which the first signal is received by the first receiver and a time at which the first signal is received by the fifth receiver;
determining an eighth time difference between a time at which the first signal is received by the second receiver and a time at which the first signal is received by the fifth receiver,
determining a ninth time difference between a time at which the first signal is received by the third receiver and a time at which the first signal is received by the fifth receiver,
determining a tenth time difference between a time at which the first signal is received by the fourth receiver and a time at which the first signal is received by the fifth receiver, and
determining the estimate of the position further based on the seventh time difference, the eighth time difference, the ninth time difference, and the tenth time difference.

6. A method as defined in claim 5, wherein the plurality of receivers comprises a sixth receiver, wherein determining the estimate of the position comprises:
determining an eleventh time difference between a time at which the first signal is received by the first receiver and a time at which the first signal is received by the sixth receiver;
determining a twelfth time difference between a time at which the first signal is received by the second receiver and a time at which the first signal is received by the sixth receiver;
determining a thirteenth time difference between a time at which the first signal is received by the third receiver and a time at which the first signal is received by the sixth receiver;
determining a fourteenth time difference between a time at which the first signal is received by the fourth receiver and a time at which the first signal is received by the sixth receiver,
determining a fifteenth time difference between a time at which the first signal is received by the fifth receiver and a time at which the first signal is received by the sixth receiver; and
determining the estimate of the position further based on the eleventh time difference, the twelfth time difference, the thirteenth time difference, the fourteenth time difference, and the fifteenth time difference.

7. A method as defined in claim 1, wherein determining the estimate of the position comprises determining the estimate of the position based on a current set of measurements and not based on previous measurements.

8. A method as defined in claim 1, wherein determining the estimate of the position comprises determining the estimate of the position based on a current set of measurements and based on previous measurements.

9. A method as defined in claim 6B, wherein determining the estimate of the position comprises determining the estimate of the position based on a model.

10. A system for facilitating surgery, the system comprising:
a surgical probe having a first transmitter coupled thereto, the first transmitter configured to transmit a first signal;
a plurality of receivers;
a position calculator operatively coupled to the plurality of receivers, wherein the position calculator is configured to determine an estimate of a position of a portion of the surgical probe based on the first signal received by the plurality of receivers;
a display unit;
a display system operatively coupled to the position calculator and the display unit, wherein the display system is configured to cause the display unit to display a representation of an anatomy of the patient and an indication of the estimate of the position, wherein the indication of the estimate of the position is integrated with the representation of the anatomy.

11. A system as defined in claim 10, wherein a second transmitter is coupled to the surgical probe, the second transmitter configured to transmit a second signal;
wherein the position calculator is configured to determine the estimate of the position further based on the second signal received by the plurality of receivers.

12. A system as defined in claim 10, wherein the plurality of receivers comprises a first receiver and a second receiver, wherein the position calculator is configured to:
determine a time difference between a time at which the first signal is received by the first receiver and a time at which the first signal is received by the second receiver; and
determine the estimate of the position based on the time difference.

13. A system as defined in claim 10, comprising a controller having a processor operatively coupled to a second memory, the plurality of the receivers, and the display unit, wherein the controller comprises the position calculator and the display system, wherein the controller is programmed to:
determine the estimate of the position of the portion of the surgical probe based on the first signal received by the plurality of receivers;
cause the display unit to display the representation of the anatomy of the patient and the indication of the estimate of the position.

14. A system as defined in claim 10, wherein the plurality of receivers are at a plurality of locations at least approximately fixed relative to a reference point;
wherein the reference point is at least approximately fixed relative to a position of the anatomy of the patient.

15. A system as defined in claim 14, wherein the plurality of receivers are coupled to a constraint system.

16. A system as defined in claim 14, wherein the plurality of receivers are coupled to an operating table.

17. A system as defined in claim 14, further comprising a head constraint system;
wherein the plurality of receivers are at a plurality of locations fixed relative to the head constraint system;
wherein the representation of the anatomy comprises a representation of at least a portion of a brain of the patient;
wherein the indication of the estimate of the position is integrated with the representation of the at least the portion of the brain.

18. A system as defined in claim 17, wherein the plurality of receivers is coupled to the head constraint system.

19. A system as defined in claim 10, further comprising signal conditioning circuitry operatively coupled to the plurality of receivers; and
at least one analog-to-digital converter operatively coupled to the signal conditioning circuitry and to the position calculator.

20. A method for configuring a system for facilitating surgery, the system to determine a position of at least a portion of a surgical probe during surgery, the method comprising the acts of:
positioning a plurality of receivers in a desired configuration;
using a transmitter and a position calculator coupled to the plurality of receivers to determine positions of the plurality of receivers;
adjusting the position calculator using the determined positions of the plurality of receivers; and
verifying an accuracy of the position calculator using a surgical probe having at least one transmitter coupled to the surgical probe.
